(19) United States
(12) Patent Application Publication
(10) Pub. No.: US 2025/0228562 A1
(43) Pub. Date: Jul. 17, 2025

(54) MOLDED BRIDGE FOR DELIVERY DEVICE

(71) Applicant: EDWARDS LIFESCIENCES CORPORATION, Irvine, CA (US)

(72) Inventor: Scott Louis Pool, Laguna Hills, CA (US)

(73) Assignee: EDWARDS LIFESCIENCES CORPORATION, Irvine, CA (US)

(21) Appl. No.: 19/097,614

(22) Filed: Apr. 1, 2025

Related U.S. Application Data
(63) Continuation of application No. PCT/US2023/034387, filed on Oct. 3, 2023.
(60) Provisional application No. 63/378,172, filed on Oct. 3, 2022.

(51) Int. Cl. A61B 17/11

(52) U.S. Cl. CPC A61B 17/11; A61B 2017/1139

(57) ABSTRACT

A delivery device for delivering a shunt device within a human body, the delivery device includes a nosecone including a straight portion and a tapered portion, and a molded bridge coupled to the nosecone. The molded bridge includes a body, a notch extending into the body of the molded bridge, and a pocket extending into the body of the molded bridge. The pocket is positioned on an opposite side of the molded bridge as the notch. The molded bridge further includes a plurality of lumens positioned within and extending at least partially through the body of the molded bridge.

### CROSS-REFERENCE TO RELATED APPLICATION(S)

This application is a continuation of International Application No. PCT/US2023/034387, filed Oct. 3, 2023, which claims the benefit of U.S. Provisional Application No. 63/378,172, filed Oct. 3, 2022, the disclosures of which are hereby incorporated by reference in their entireties.

### BACKGROUND

The present disclosure relates generally to implantable devices for a human body, and more particularly to a delivery device used to aid in delivering and placing a shunt device within a human body.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt device can be positioned in the septal wall between the left atrium and the right atrium to shunt blood from the left atrium into the right atrium, thus reducing the pressure in the left atrium.

### SUMMARY

A delivery device for delivering a shunt device within a human body, the delivery device includes a nosecone including a straight portion and a tapered portion, and a molded bridge coupled to the nosecone. The molded bridge includes a body, a notch extending into the body of the molded bridge, and a pocket extending into the body of the molded bridge. The pocket is positioned on an opposite side of the molded bridge as the notch. The molded bridge further includes a plurality of lumens positioned within and extending at least partially through the body of the molded bridge.

A method of using a delivery device to deliver a shunt device within a human body includes inserting a guidewire into the human body, and inserting an end of the guidewire positioned outside the human body into an aperture extending through a nosecone of the delivery device and a guidewire lumen of a molded bridge of the delivery device. The delivery device is translated along the guidewire to position the delivery device within the human body. A moveable-arm wire is removed from a moveable-arm lumen extending through the molded bridge to release a first proximal arm of the shunt device from the delivery device. A release wire is removed from a release wire lumen extending through the molded bridge to release a second proximal arm of the shunt device from the delivery device.

### BRIEF DESCRIPTION OF THE DRAWINGS

### Anatomy of Heart H and Vasculature V

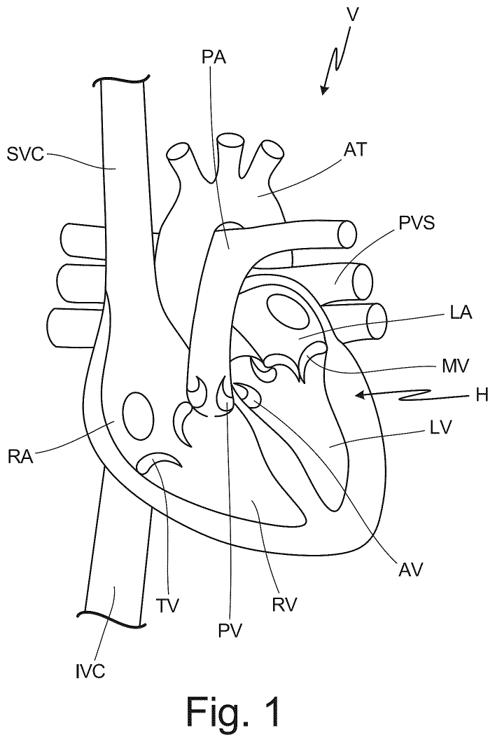

FIG. 1 is a schematic diagram of a heart and vasculature.

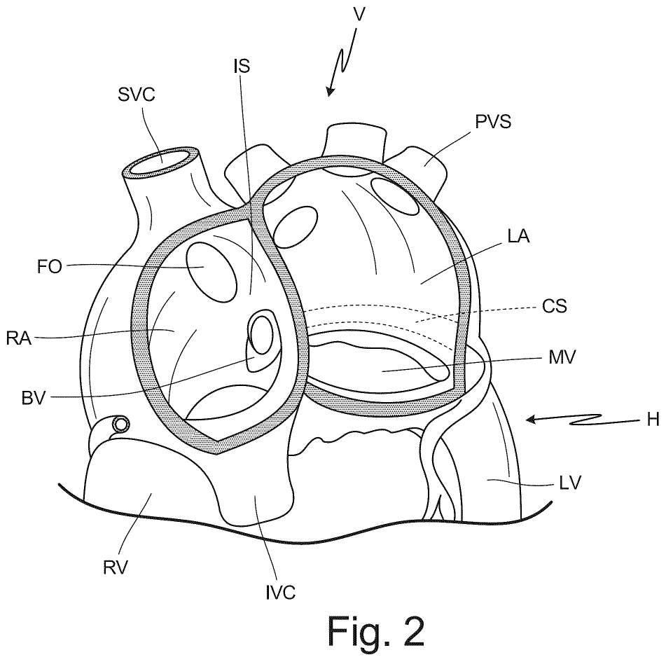

FIG. 2 is a schematic cross-sectional view of the heart.

### Shunt Devices 100 and 100′

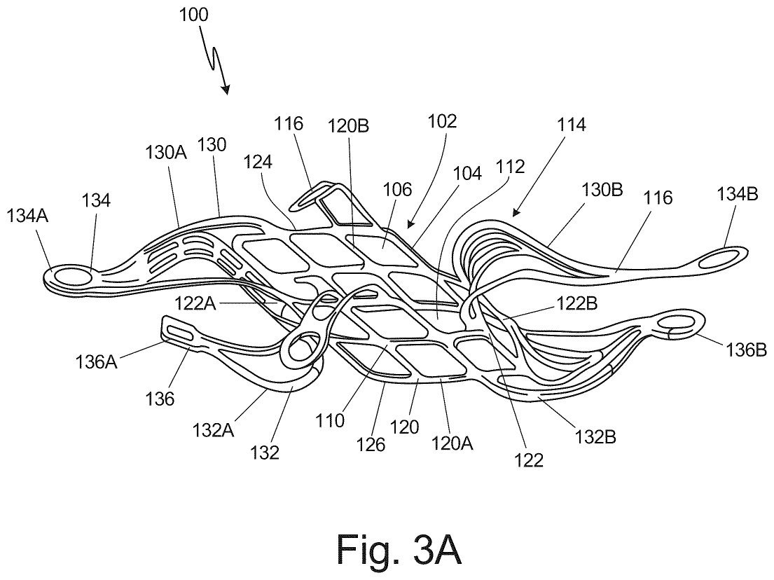

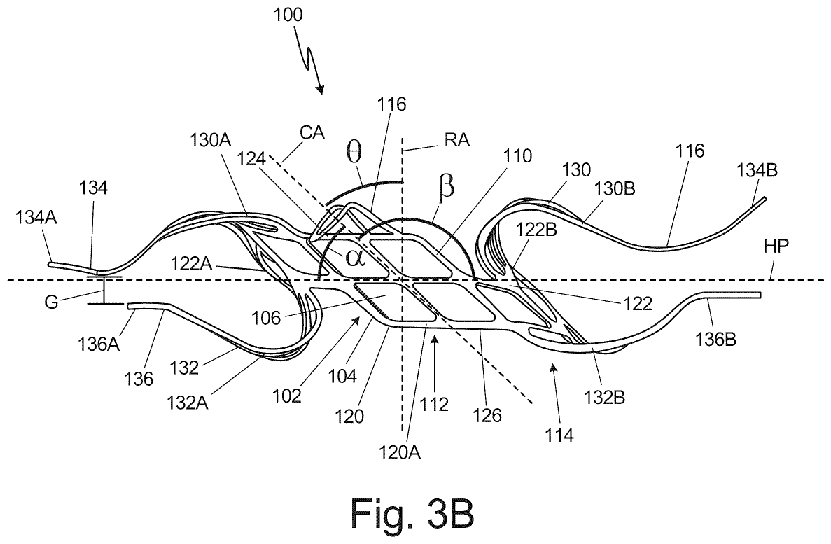

FIG. 3A is a perspective view of a shunt device.

FIG. 3B is a side view of the shunt device.

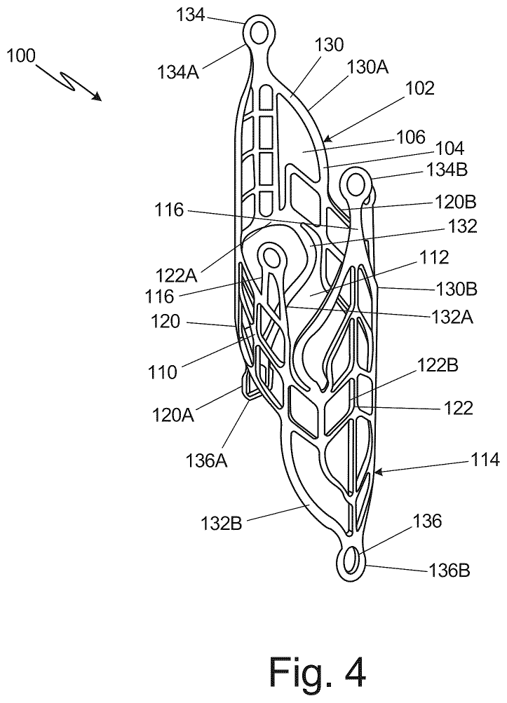

FIG. 4 is a perspective view of the shunt device in a collapsed configuration.

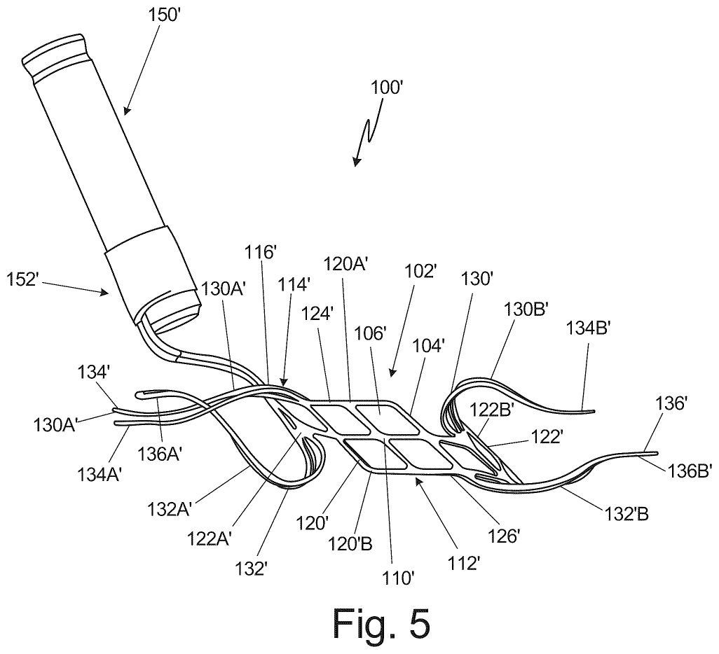

FIG. 5 is a perspective view of a shunt device including a sensor.

### Delivery Catheter 200

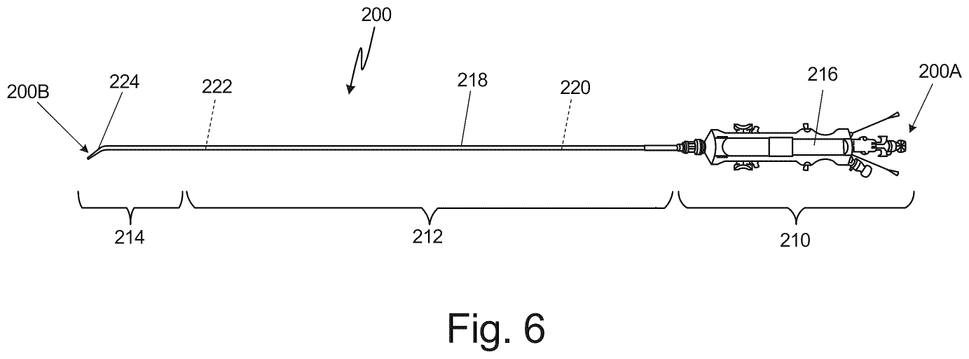

FIG. 6 is a side view of a delivery catheter.

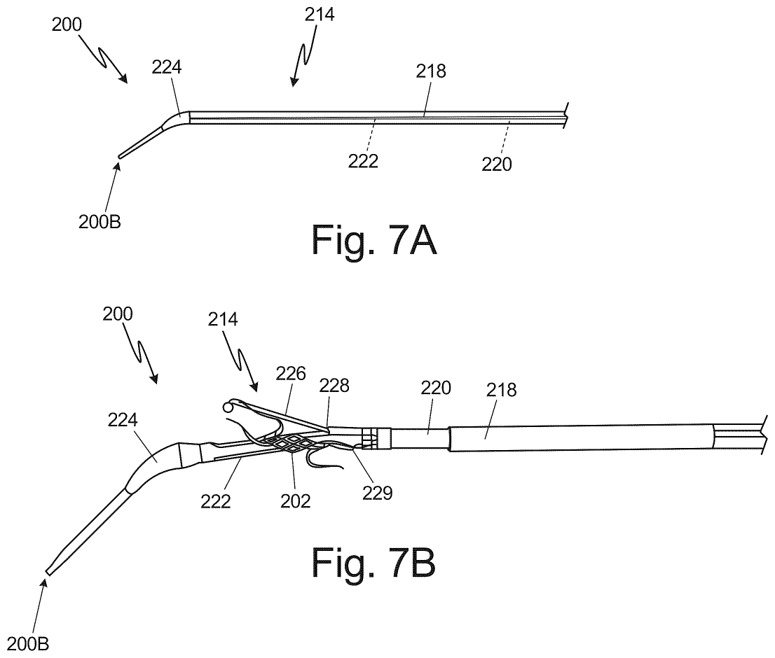

FIG. 7A is a side view of a distal portion of the delivery catheter in a sheathed state.

FIG. 7B is a side view of the distal portion of the delivery catheter in an unsheathed state.

### Delivery Method 300

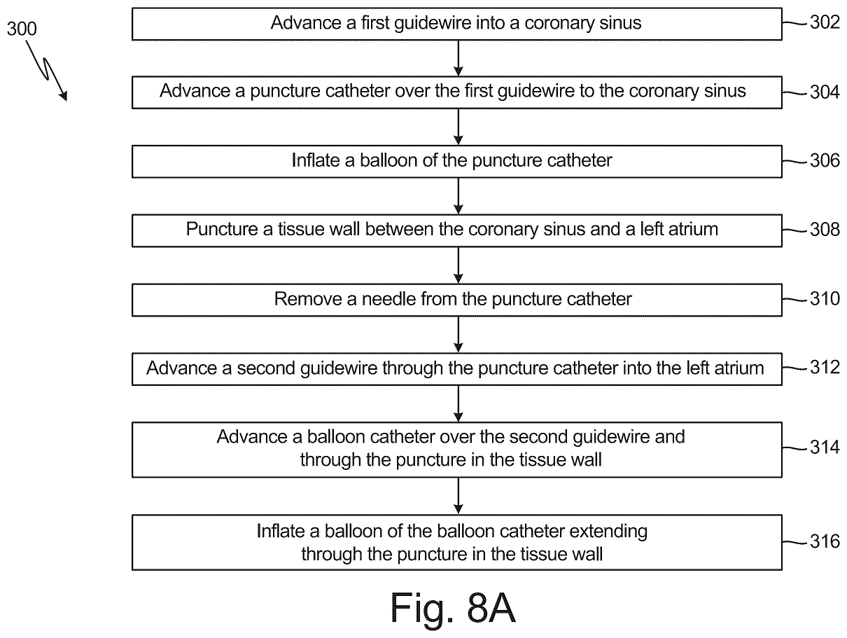

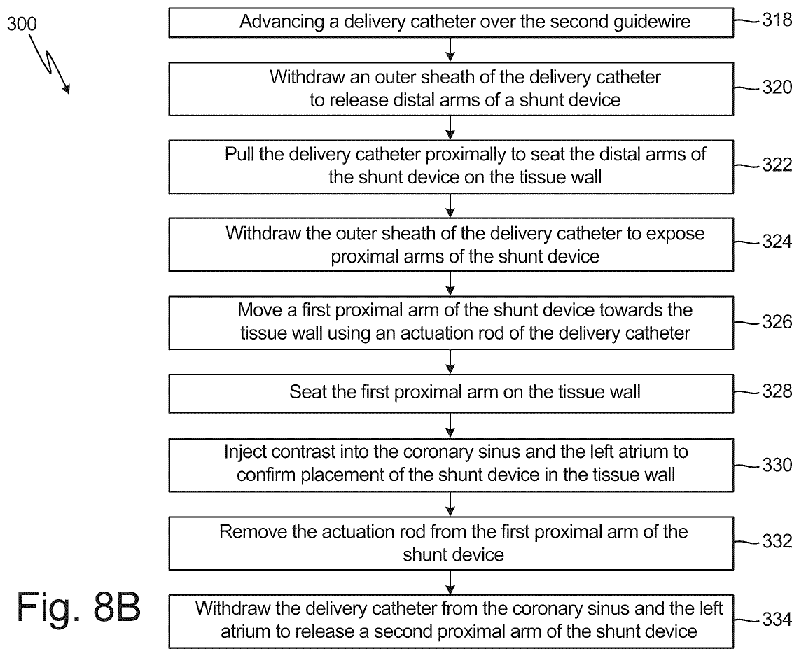

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

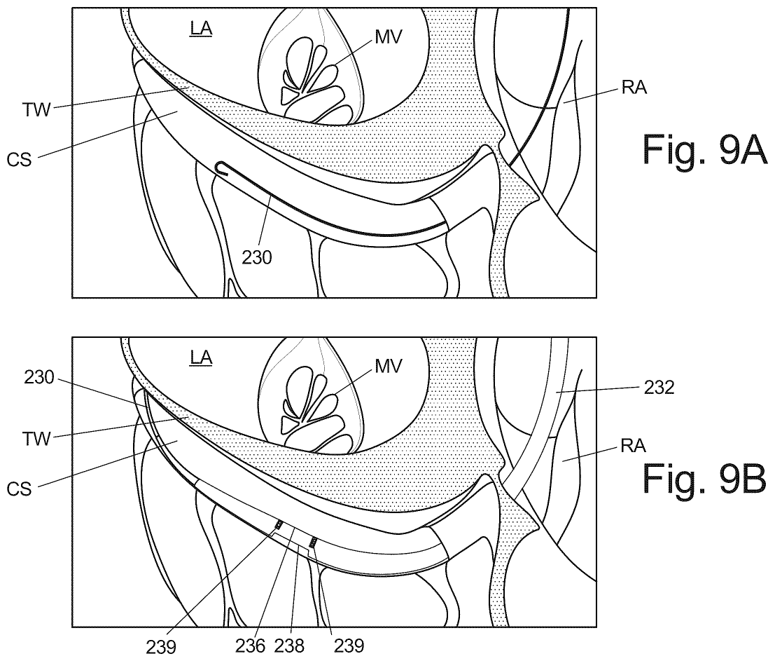

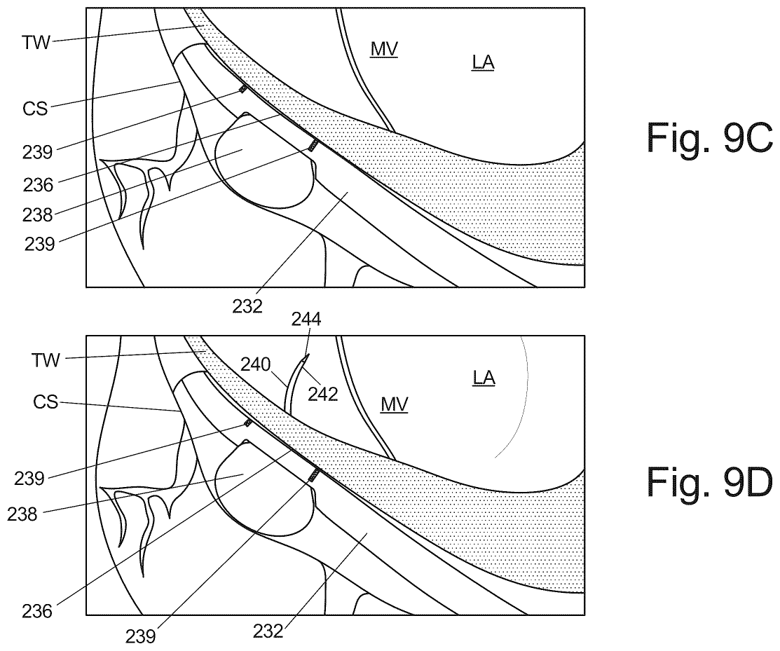

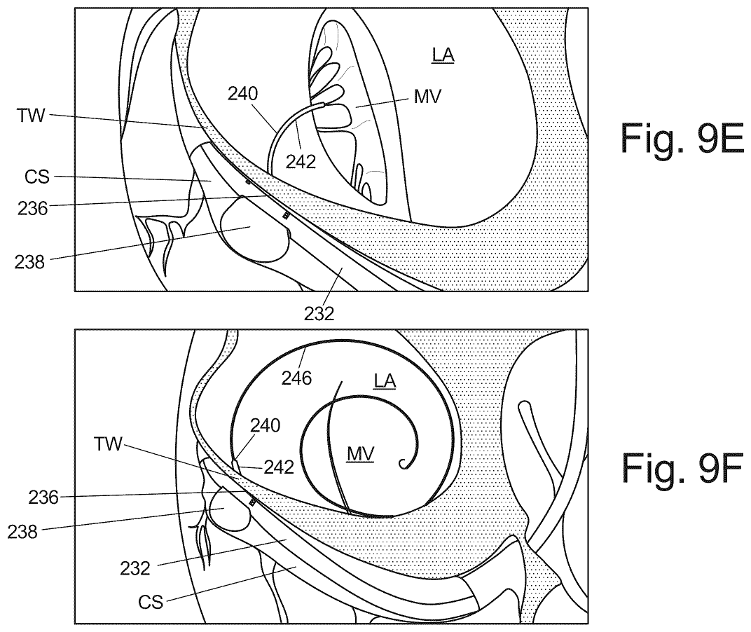

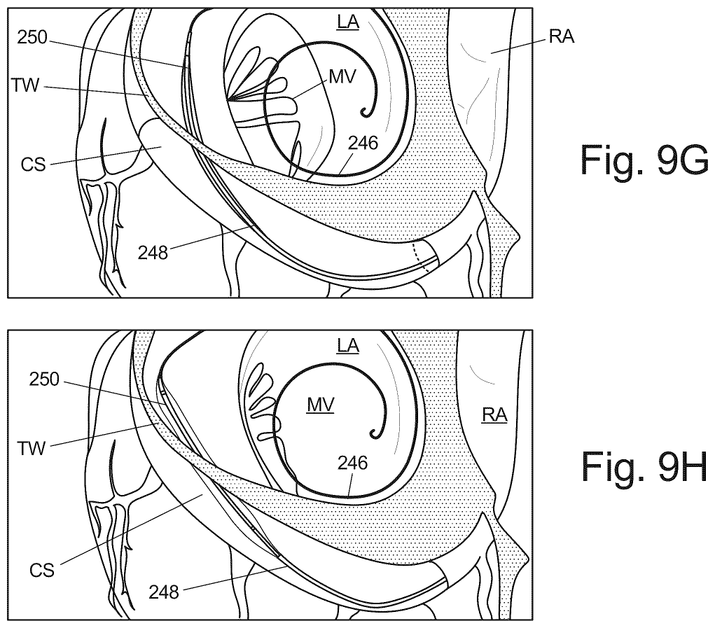

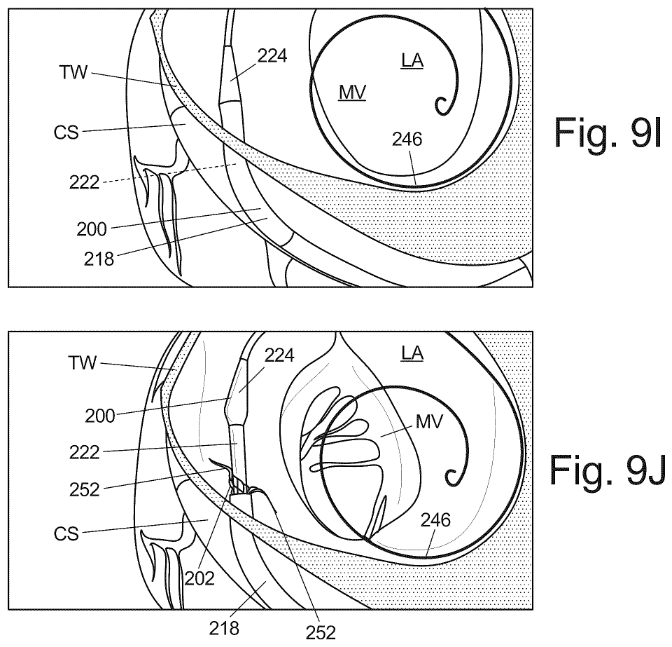

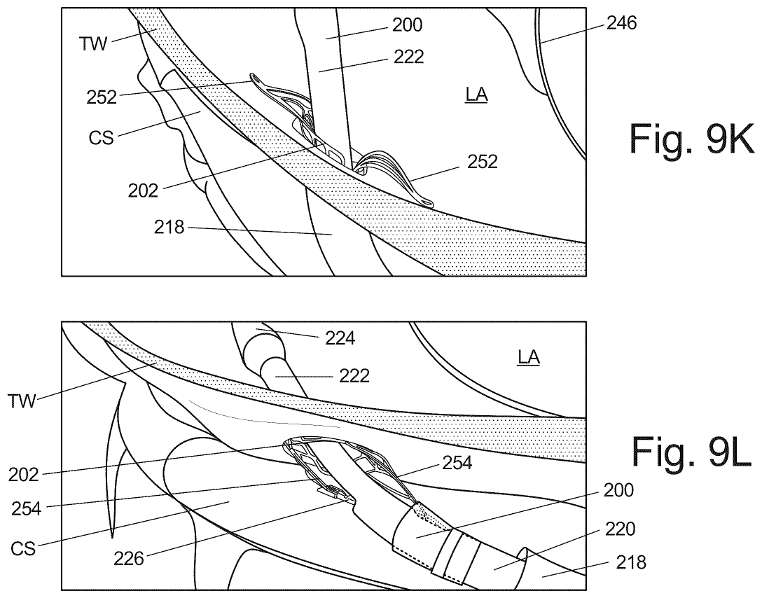

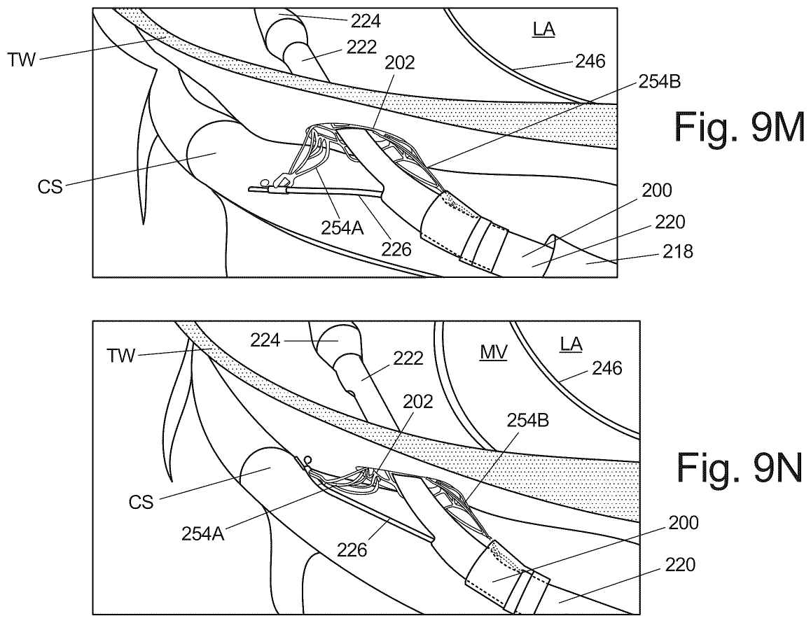

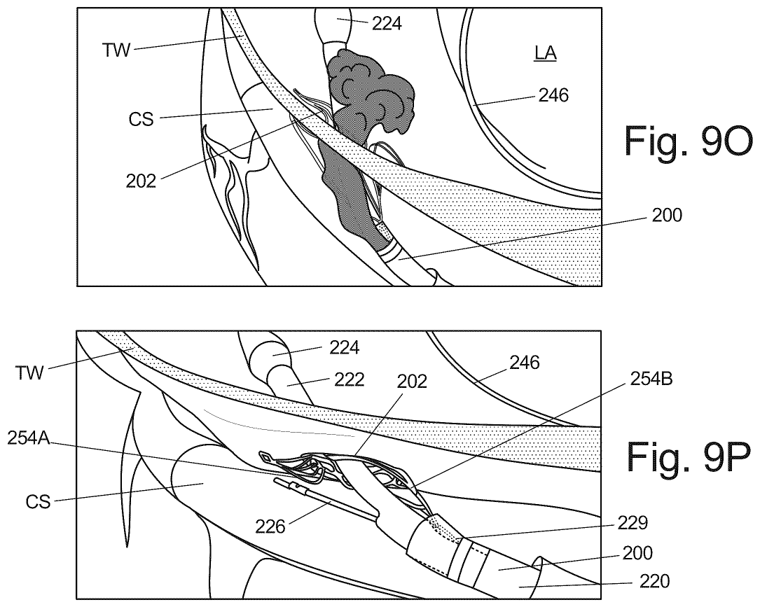

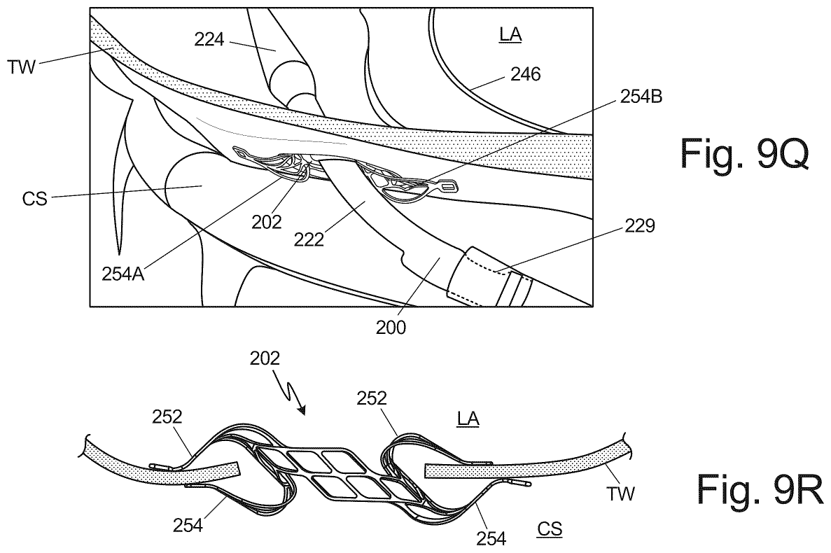

FIGS. 9A-9R are schematic views showing the steps for implanting a shunt device in the tissue wall between the coronary sinus and the left atrium.

### Delivery Device 1100

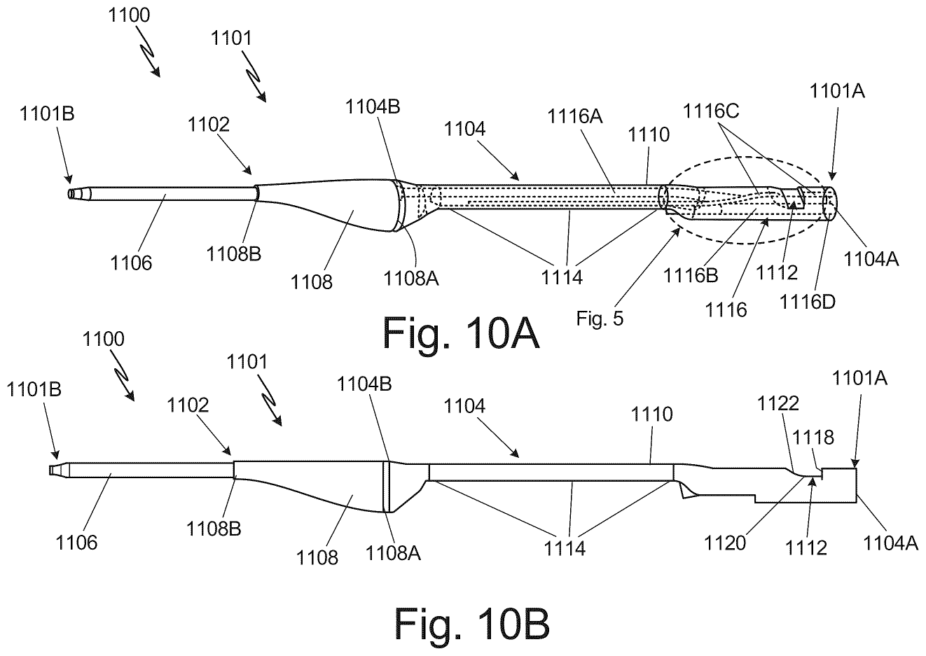

FIG. 10A is a perspective view of a distal portion of a delivery device for delivering and placing a shunt device within a human body.

FIG. 10B is a side view of the distal portion of the delivery device of FIG. 10A.

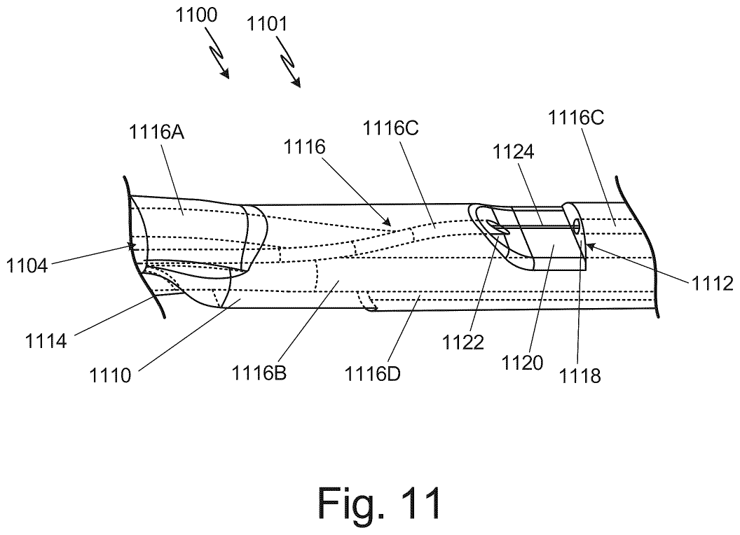

FIG. 11 is a detailed perspective view of part of the distal portion of the delivery device, shown with a dashed oval in FIG. 10A.

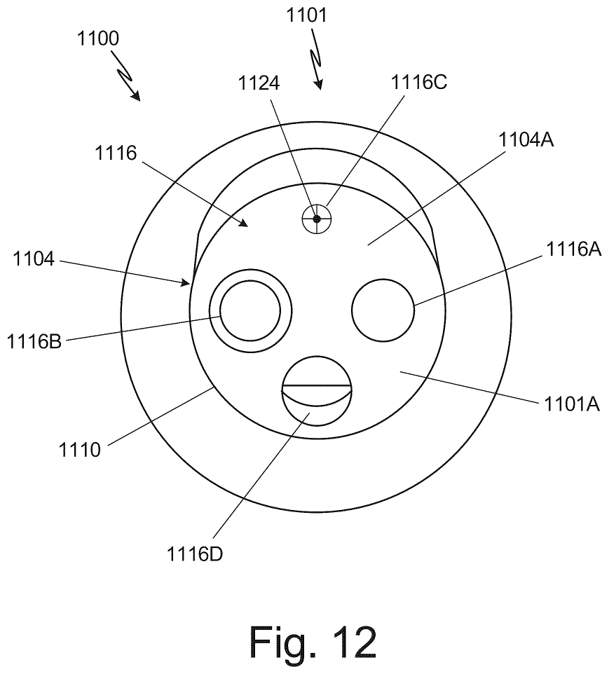

FIG. 12 is an end view of a proximal end of a molded bridge of the distal portion of the delivery device.

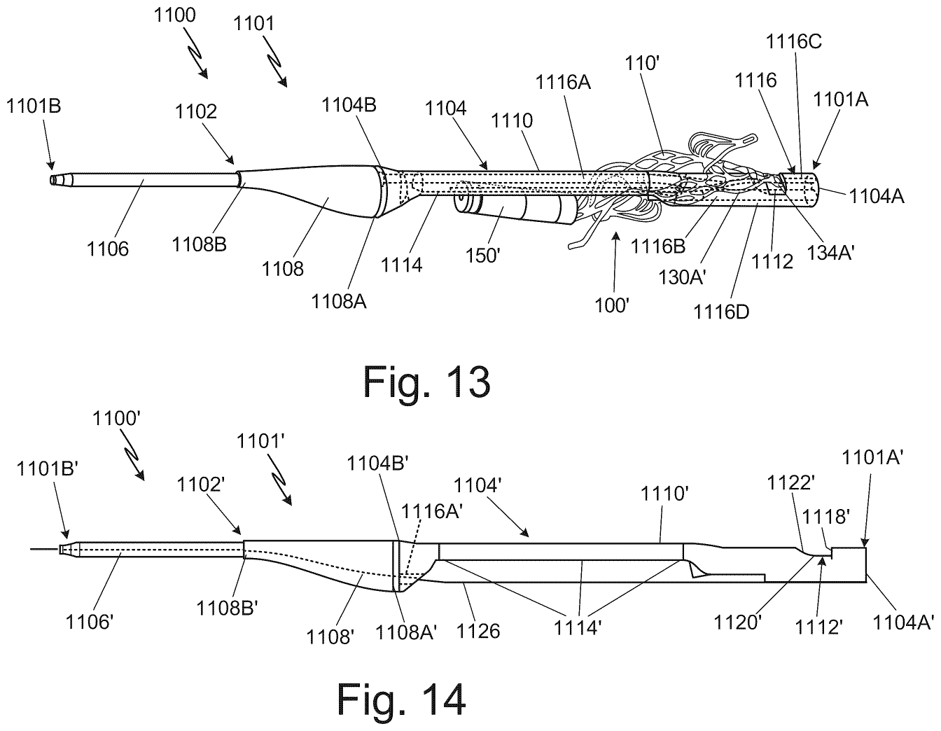

FIG. 13 is a perspective view of the distal portion of the delivery device including an attached shunt device.

FIG. 14 is a side view of another example of a distal portion of a delivery device for delivering and placing a shunt device within a human body.

### DETAILED DESCRIPTION

### Anatomy of Heart H and Vasculature V (FIGS. 1-2)

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees) (<90° and second angle β can be greater than ninety degrees) (>90°. In other examples, first angle α and second angle β can be any suitable combination of angles that add to one hundred eighty degrees) (180°.The difference between first angle α and second angle β (and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′ (including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9R.

### Delivery Method 300 (FIGS. 8A-9R)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9R are schematic views showing the steps for implanting shunt device 202 in tissue wall TW between coronary sinus CS and left atrium LA. FIGS. 8A-9R will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 91) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes injecting contrast into coronary sinus CS and left atrium LA to confirm placement of shunt device 202 in tissue wall TW, as shown in FIG. 90. Contrast can be injected through a lumen extending through delivery catheter 200. The contrast can move through coronary sinus CS and left atrium LA. The contrast will highlight shunt device 202 under fluoroscopy to confirm proper placement of distal arms 252 and first proximal arm 254A of shunt device 202 on tissue wall TW.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 9R. Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9R, but can be a single catheter in alternate examples.

### Delivery Device 1100 (FIGS. 10A-13)

FIG. 10A is a perspective view of distal portion 1101 of delivery device 1100 for delivering and placing shunt device 100 within a human body. FIG. 10B is a side view of distal portion 1101 of delivery device 1100. FIGS. 10A-10B will be discussed together. Delivery device 1100 includes distal portion 1101 (having proximal end 1101A and distal end 1101B), nosecone 1102, molded bridge 1104 (having proximal end 1104A and distal end 1104B), straight portion 1106, tapered portion 1108 (having proximal end 1108A and distal end 1108B), body 1110, notch 1112, pocket 1114, plurality of lumens 1116 (including first lumen 1116A, second lumen 1116B, third lumen 1116C, and fourth lumen 1116D), vertical portion 1118, horizontal portion 1120, and angled portion 1122.

Delivery device 1100 is a component and/or an assembly that is configured to be transported into and out of a human body. Delivery device 1100 is one example of a delivery catheter, for example delivery catheter 200, that can be used to implant a shunt device. More specifically, delivery device 1100 is configured to transport and place shunt device 100 as shown in FIGS. 3A-4 or shunt device 100′ as shown in FIG. 5 within a human body. The description herein will describe shunt device 100 for clarity. However, shunt device 100 is one example of a shunt device that can be implanted using delivery device 1100, and delivery device 1100 can be used to implant any suitable shunt device in alternate examples. After shunt device 100 has been deployed, delivery device 1100 is removed from the human body. In some examples, shunt device 100 is coupled to delivery device 1100 and delivery device 1100 is translated along a guidewire into a human body to properly position shunt device 100 within the human body. In other examples, features of delivery device 1100 can aid in releasing shunt device 100 from the delivery device 1100, allowing shunt device 100 to secure to tissue within the human body. The various examples, features, and methods of using delivery device 1100 will be described in detail below.

In the example shown in FIGS. 10A-10B, distal portion 1101 of delivery device 1100 includes proximal end 1101A, distal end 1101B, nosecone 1102, and molded bridge 1104. Proximal end 1101A of distal portion 1101 of delivery device 1100 is the end of delivery device 1100 closest to a user's hand when delivery device 1100 is initially inserted or translated into a human body. Distal end 1101B of delivery device 1100 is the end of delivery device 1100 furthest from a user's hand when delivery device 1100 is initially inserted into a human body.

Nosecone 1102 is positioned distally to molded bridge 1104. Nosecone 1102 includes straight portion 1106 and tapered portion 1108. Straight portion 1106 extends from distal end 1101B of distal portion 1101 to tapered portion 1108. Tapered portion 1108 includes proximal end 1108A and distal end 1108B. Tapered portion 1108 extends from straight portion 1106 to proximal end 1101A of distal portion 1101.

Molded bridge 1104 includes proximal end 1104A, distal end 1104B, body 1110, notch 1112, pocket 1114, and a plurality of lumens 1116. In some examples, the plurality of lumens 1116 can include first lumen 1116A, second lumen 1116B, third lumen 1116C, and fourth lumen 1116D. In some examples, notch 1112 includes vertical portion 1118, horizontal portion 1120, and angled portion 1122. In some examples, distal portion 1101 of delivery device 1100 can be an assembly in which multiple components are coupled together to form distal portion 1101 of delivery device 1100. In other examples, distal portion 1101 of delivery device 1100 can be a molded component in which each of the individual features of distal portion 1101 of delivery device 1100 are a single, unitary component.

As shown in FIGS. 10A-10B, nosecone 1102 includes straight portion 1106 and tapered portion 1108. Straight portion 1106 is positioned adjacent and closest to distal end 1101B of distal portion 1101 of delivery device 1100, compared to any other feature/component of delivery device 1100. Straight portion 1106 includes a generally constant diameter extending a length of straight portion 1106 and straight portion 1106 includes a lumen extending fully through straight portion 1106. In some examples, as shown, an end of straight portion 1106 closest to distal end 1101B of distal portion 1101 of delivery device 1100 can include a portion with a reduced diameter. In other examples, the outer diameter of straight portion 1106 can be constant along an entire length of straight portion 1106. In some examples, as shown, an end of straight portion 1106 can be coupled to an end of tapered portion 1108. In other examples, straight portion 1106 and tapered portion 1108 can be formed as a unitary, single-piece component. In some examples, straight portion 1106 and tapered portion 1108 can each be constructed from one or more of a thermoplastic polymer, a nylon, or other polymer based material.

Tapered portion 1108 is positioned between and coupled to both straight portion 1106 and molded bridge 1104. Tapered portion 1108 includes proximal end 1108A positioned at one end of tapered portion 1108 and distal end 1108B positioned at the opposite end of tapered portion 1108. More specifically, proximal end 1108A of tapered portion 1108 is coupled to distal end 1104B of molded bridge 1104, and distal end 1108B of tapered portion 1108 is coupled to straight portion 1106. Tapered portion 1108 of nosecone 1102 also includes an aperture extending fully through tapered portion 1108 from proximal end 1108A to distal end 1108B. The aperture extending through tapered portion 1108 is axially aligned with the aperture extending through straight portion 1106 of nosecone 1102. Further, the apertures extending through straight portion 1106 and tapered portion 1108 are configured to accept a guidewire, discussed further below.

Proximal end 1108A of tapered portion 1108 include a first outer diameter and distal end 1108B of tapered portion 1108 includes a second outer diameter. In the example shown, the first outer diameter of proximal end 1108A is greater than the second outer diameter of distal end 1108B. In some examples, the first outer diameter of proximal end 1108A can range between 3 millimeters (0.118 inches) and 9 millimeters (0.354 inches). Tapered portion 1108 includes distal end 1108B with a smaller outer diameter to facilitate deployment of shunt device 100 within a human body, discussed further below. Further, a central axis of distal end 1108B is offset from a central axis of proximal end 1108A to facilitate translation of shunt device 100 within a human body. In other words, the central axis of distal end 1108B is not axially aligned with the central axis of proximal end 1108A to aid in translation of shunt device 100 within the human body, as discussed in reference to FIG. 13 below.

Molded bridge 1104 includes proximal end 1104A and distal end 1104B. Proximal end 1104A of molded bridge 1104 is positioned adjacent proximal end 1101A of distal portion 1101 of delivery device 1100. Distal end 1104B of molded bridge 1104 is positioned at an opposite end of molded bridge 1104 as proximal end 1104A. Further, distal end 1104B of molded bridge 1104 is positioned closest to distal end 1101B of distal portion 1101 of delivery device 1100, compared to any other feature of molded bridge 1104. Distal end 1104B of molded bridge 1104 is coupled to proximal end 1108A of tapered portion 1108 of nosecone 1102. In some examples, distal end 1104B of molded bridge 1104 coupled to proximal end 1108A of tapered portion 1108 includes an outer diameter that is equal to the first outer diameter of proximal end 1108A of tapered portion 1108. In other examples, distal end 1104B of molded bridge 1104 may not have the same outer diameter as proximal end 1108A of tapered portion 1108.

Body 1110 is the main structure of molded bridge 1104 in which other features of molded bridge 1104 are positioned. In some examples, body 1110 can be constructed from one or more of a thermoplastic elastomer, a nylon, or other polymer blend material. Further, in some examples, body 1110 of molded bridge 1104 can be constructed from the same material as nosecone 1102. Body 1110 has a generally elongated cylindrical shape with cutouts extending into body 1110. In some examples, notch 1112 extends into body 1110 of molded bridge 1104. In further examples, pocket 1114 extends into body 1110 of molded bridge 1104 such that pocket 1114 is positioned on an opposite side of molded bridge 1104 as notch 1112. Further, in some examples, a plurality of lumens 1116 are positioned within and extend at least partially through body 1110 of molded bridge 1104. Notch 1112 and plurality of lumens 1116 will be discussed in greater detail below with respect to FIG. 11.

FIG. 11 is a detailed perspective view of part of distal portion 1101 of delivery device 1100, shown with a dashed oval in FIG. 10A. Delivery device 1100 includes distal portion 1101, molded bridge 1104, body 1110, notch 1112, pocket 1114, plurality of lumens 1116 (including first lumen 1116A, second lumen 1116B, third lumen 1116C, and fourth lumen 1116D), vertical portion 1118, horizontal portion 1120, angled portion 1122, and release wire 1124.

Notch 1112 of molded bridge 1104 is positioned near proximal end 1104A (shown in FIGS. 10A-10B) of molded bridge 1104 on a top side of molded bridge 1104. In some examples, notch 1112 is configured to seat an arm of shunt device 100, discussed further below with respect to FIG. 13. In the example shown in FIG. 11, notch 1112 includes vertical portion 1118, horizontal portion 1120, and angled portion 1122. Vertical portion 1118 of notch 1112 is positioned closest to proximal end 1104A of molded bridge 1104 compared to horizontal portion 1120 and angled portion 1122. Vertical portion 1118 is a flat surface that extends a partial distance from an outer surface of molded bridge 1104 towards a central axis of molded bridge 1104. Horizontal portion 1120 of notch 1112 extends between vertical portion 1118 and angled portion 1122. More specifically, horizontal portion 1120 is a flat surface that is oriented orthogonal to vertical portion 1118, and horizontal portion 1120 extends from an edge of vertical portion 1118 towards distal end 1104B (shown in FIGS. 10A-10B) of molded bridge 1104 to connect vertical portion 1118 to angled portion 1122. Angled portion 1122 of notch 1112 is positioned closest to distal end 1104B of molded bridge 1104 compared to vertical portion 1118 and horizontal portion 1120. More specifically, angled portion 1122 extends from an end of horizontal portion 1120 at an oblique angle towards distal end 1104B of molded bridge 1104. Collectively, vertical portion 1118, horizontal portion 1120, and angled portion 1122 are configured to seat an arm of shunt device 100, discussed further below. Release wire 1124 extends across notch 1112.

Referring again to FIGS. 10A-10B, pocket 1114 of molded bridge 1104 is positioned on a bottom side of molded bridge 1104, opposite notch 1112 positioned on a top side of molded bridge 1104. Further, pocket 1114 extends a partial distance between proximal end 1104A and distal end 1104B of molded bridge 1104. More specifically, pocket 1114 is positioned closer to distal end 1104B than proximal end 1104A of molded bridge 1104, and pocket 1114 extends a partial distance towards proximal end 1104A but does not extend all the way to proximal end 1104A. Pocket 1114 is a semi-circular indent or cutout within body 1110 of molded bridge 1104 and extends from a bottom side of molded bridge 1104 towards a central axis of molded bridge 1104. As shown best in FIG. 13, pocket 1114 is configured to seat an arm and a portion of central flow tube 110 of shunt device 100. Further, in some examples, pocket 1114 can be configured to seat sensor 150′ coupled to shunt device 100′ (shown in FIG. 5), discussed in more detail with respect to FIG. 13.

In some examples, molded bridge 1104 can include a plurality of lumens 1116 positioned within and extending at least partially through body 1110 of molded bridge 1104. Each of the plurality of lumens 1116 can be tubular channels or passages that extend at least partially through body 1110 of molded bridge 1104. In one example, each of the plurality of lumens can have differing lengths. As shown best in FIGS. 11-12, the plurality of lumens 1116 can include first lumen 1116A, second lumen 1116B, third lumen 1116C, and fourth lumen 1116D.

FIG. 12 is an end view of proximal end 1104A of molded bridge 1104 of distal portion 1101 of delivery device 1100. Delivery device 1100 includes distal portion 1101 (having proximal end 1101A), molded bridge 1104 (having proximal end 1104A), body 1110, plurality of lumens 1116 (including first lumen 1116A, second lumen 1116B, third lumen 1116C, and fourth lumen 1116D), and release wire 1124.

Plurality of lumens 1116 are shown in FIGS. 10A and 11-12. Plurality of lumens 1116 includes each of first lumen 1116A, second lumen 1116B, third lumen 1116C, and fourth lumen 1116D. In the example shown in FIGS. 10A-12, each of first lumen 1116A, second lumen 1116B, third lumen 1116C, and fourth lumen 1116D can have circular cross-sections when viewing in the axial direction. In other examples, each of first lumen 1116A, second lumen 1116B, third lumen 1116C, and fourth lumen 1116D can have a cross-section of any geometric shape. In the example shown in FIGS. 10A-12, first lumen 1116A, second lumen 1116B, third lumen 1116C, and fourth lumen 1116D can have different diameters. In alternate examples, first lumen 1116A, second lumen 1116B, third lumen 1116C, and fourth lumen 1116D can have equal diameters.

As shown best in FIG. 10A, first lumen 1116A fully extends through body 1110 from proximal end 1104A to distal end 1104B of molded bridge 1104. Further, first lumen 1116A extends through proximal end 1104A and distal end 1104B of molded bridge 1104. The distal end of first lumen 1116A at distal end 1104B of molded bridge 1104 is axially aligned with the apertures extending through both straight portion 1106 and tapered portion 1108 of nosecone 1102. The proximal end of first lumen 1116A at proximal end 1104A of molded bridge 1104 is axially aligned with a lumen extending through an inner sheath (not shown in FIGS. 10A-12) of delivery device 1100. Further, first lumen 1116A includes a curve or bend along the length of first lumen 1116A such that the proximal end of first lumen 1116A extending through proximal end 1104A is not axially aligned with the distal end of first lumen 1116A extending through distal end 1104B. First lumen 1116A is curved between notch 1112 and pocket 114 of molded bridge 1104. First lumen 1116A can include a curve or bend to ensure each of first lumen 1116A, second lumen 1116B, third lumen 1116C, and fourth lumen 1116D can fit within body 1110 of molded bridge 1104 without interfering with each other or with notch 1112 or pocket 1114.

First lumen 1116A is configured to accept and receive a guidewire fully extending through first lumen 1116A. As such, a guidewire can be inserted through the apertures within straight portion 1106 and tapered portion 1108 of nosecone 1102, and then the guidewire can be inserted into first lumen 1116A and pushed through first lumen 1116A until the guidewire exits first lumen 1116A adjacent proximal end 1104A of molded bridge 1104. In some examples, the guidewire can be a thin wire used to guide the placement of delivery device 1100 within a human body during a medical procedure. As such, first lumen 1116A can also be referred to as a guidewire lumen because first lumen 1116A accepts a guidewire, and delivery device 1100 utilizes first lumen 1116A to translate delivery device 1100 along a guidewire into a human body. As discussed with respect to FIG. 14, in some examples, portions of first lumen 116A can be eliminated and the guidewire can extend along an external surface of molded bridge 1104.

Referring to FIGS. 10A and 11, second lumen 1116B extends through body 1110 of molded bridge 1104 from proximal end 1104A to pocket 1114 of molded bridge 1104. Further, second lumen 1116B extends through proximal end 1104A and extends through body 1110 adjacent and terminating at the end of pocket 1114 positioned closest to proximal end 1104A of molded bridge 1104. A proximal end of second lumen 1116B is aligned with a lumen extending through an inner sheath (not shown in FIGS. 10A-12) of delivery device 1100. In the example shown in FIGS. 10A-12, second lumen 1116B is a straight lumen extending through body 1110 of molded bridge 1104. In alternate examples, second lumen 1116B can be a curved lumen extending through body 1110 of molded bridge 1104.

Second lumen 1116B is configured to provide a flow path for contrast agent/dye to flow through molded bridge 1104 for delivery into the human body. As such, second lumen 1116B can also be referred to as the contrast lumen. The contrast agent/dye can flow into second lumen 1116B at proximal end 1104A of molded bridge 1104, the contrast agent/dye can flow through second lumen 1116B towards distal end 1104B of molded bridge 1104, and then the contrast agent/dye can flow out from second lumen 1116B adjacent pocket 1114 of molded bridge 1104.

Flowing the contrast agent/dye through second lumen 1116B, after delivery device 1100 is within a human body, can aid a doctor or user in ensuring proper placement of delivery device 1100 and shunt device 100 within a human body.

Referring to FIGS. 10A and 11, third lumen 1116C extends through body 1110 of molded bridge 1104 from proximal end 1104A of molded bridge 1104 to notch 1112, and then third lumen 1116C includes a break or gap and third lumen 1116C resumes on an opposite side of notch 1112. More specifically, a first section of third lumen 1116C extends through body 1110 from proximal end 1104A of molded bridge 1104 to vertical portion 1118 of notch 1112, and third lumen 1116C extends through vertical portion 1118 of notch 1112. A break or gap is present in third lumen 1116C at notch 1112 and a second section of third lumen 1116C extends through angled portion 1122 of notch 1112 and extends through body 1110 in the direction of distal end 1104B of molded bridge 1104. The second section of third lumen 1116C does not extend all the way to distal end 1104B of molded bridge 1104.

As shown best in FIG. 11, third lumen 1116C is configured to accept and receive release wire 1124 extending within and at least partially through third lumen 1116C. As such, third lumen 1116C can also be referred to as a release wire lumen. Release wire 1124 can extend through third lumen 1116C and distal arm 130 or proximal arm 132 of shunt device 100 (shown in FIGS. 3A-4), depending on the orientation of shunt device 100, to secure and hold shunt device 100 to delivery device 1100. Third lumen 1116C is split over notch 1112 to allow release wire 1124 to extend through distal arm 130 or proximal arm 132 of shunt device 100 which is positioned in notch 1112. Further, a user can remove release wire 1124 from third lumen 1116C and distal arm 130 or proximal arm 132 to allow shunt device 100 to release from delivery device 1100 to be secured to tissue within a human body, discussed in more detail with respect to FIG. 13.

As shown best in FIG. 11, fourth lumen 1116D extends through body 1110 of molded bridge 1104 from proximal end 1104A of molded bridge 1104 to pocket 1114 of molded bridge 1104. Further, fourth lumen 1116D extends through proximal end 1104A and extends through body 1110 adjacent and terminating at the end of pocket 1114 positioned closest to proximal end 1104A of molded bridge 1104. In the example shown in FIGS. 10A-12, fourth lumen 1116D is a straight lumen extending through body 1110 of molded bridge 1104. In alternate examples, fourth lumen 1116D can be a curved lumen extending through body 1110 of molded bridge 1104.

Fourth lumen 1116D is configured to accept and receive a moveable-arm wire fully extending through fourth lumen 1116D. As such, in some examples, fourth lumen 1116D can also be referred to as a moveable-arm wire lumen. A moveable-arm wire can extend through fourth lumen 1116D and distal arm 130 or proximal arm 132 of shunt device 100, depending on the orientation of shunt device 100, to secure and hold shunt device 100 to delivery device 1100. Further, a user can remove the moveable-arm wire from fourth lumen 1116D and distal arm 130 or proximal arm 132 to allow shunt device 100 to release from delivery device 1100 to be secured to tissue within a human body, discussed in more detail with respect to FIG. 13.

Each of first lumen 1116A, second lumen 1116B, third lumen 1116C, and fourth lumen 1116D are configured to aid in delivering and placing shunt device 100 within a human body. Further, each of first lumen 1116A, second lumen 1116B, third lumen 1116C, and fourth lumen 1116D are configured to serve distinct purposes during the delivery and placement of shunt device 100. Although first lumen 1116A, second lumen 1116B, third lumen 1116C, and fourth lumen 1116D are shown having a specific shape and configuration, it is to be understood that the shape and configuration of each of first lumen 1116A, second lumen 1116B, third lumen 1116C, and fourth lumen 1116D could be different than what is shown without deviating from their respective intended purposes.

In some examples, molded bridge 1104 can be manufactured using an injection molding process. In such examples, each of first lumen 1116A, second lumen 1116B, third lumen 1116C, and fourth lumen 1116D can be individually surrounded by an outer casing (not shown), defining the shape of each of first lumen 1116A, second lumen 1116B, third lumen 1116C, and fourth lumen 1116D. The outer casings surrounding each of first lumen 1116A, second lumen 1116B, third lumen 1116C, and fourth lumen 1116D can be constructed from a material having a higher durometer (harder material) than the material used to construct body 1110 of molded bridge 1104. The outer casings having a higher durometer can prevent deformation of the lumens during the injection molding process, ensuring the lumens within body 1110 of molded bridge 1104 have the desired shape and configuration. In other examples, each of first lumen 1116A, second lumen 1116B, third lumen 1116C, and fourth lumen 1116D may not be surrounded by an outer casing. Further, in some examples, a portion near distal end 1101B of distal portion 1101 of delivery device 1100 can be constructed from a polymer having a first durometer hardness and a portion near proximal end 1101A of distal portion 1101 of delivery device 1100 can be constructed from a polymer having a second durometer hardness. The first durometer hardness of the portion near distal end 1101B can be less than the second durometer hardness of the portion near proximal end 1101A to facilitate the bending and tracking of delivery device 1100 along a guidewire during the insertion and removal of delivery device 1100. More specifically, a softer (or less hard) material near distal end 1101B reduces the amount of frictional force experienced by delivery device 1100 during the insertion and removal of delivery device 1100, allowing delivery device 1100 to easily be inserted and removed from a human body.

FIG. 13 is a perspective view of distal portion 1101 of delivery device 1100 including an attached shunt device 100′. Delivery device 1100 includes distal portion 1101 (having proximal end 1101A and distal end 1101B), nosecone 1102, molded bridge 1104 (having proximal end 1104A and distal end 1104B), straight portion 1106, tapered portion 1108 (having proximal end 1108A and distal end 1108B), body 1110, notch 1112, pocket 1114, and plurality of lumens 1116 (including first lumen 1116A, second lumen 1116B, third lumen 1116C, and fourth lumen 1116D). FIG. 13 also shows shunt device 100′, which includes central flow tube 110′, distal arm 130A′, terminal end 136A′, and sensor 150′.

Although the following discussion specifically describes shunt device 100′ with sensor 150′ coupled to delivery device 1100, it is to be understood that the following discussion equally applies to shunt device 100 (without a sensor) coupled to delivery device 1100. To avoid redundancy, only a discussion regarding shunt device 100′ with sensor 150′ coupled to delivery device 1100 will be discussed. As shown, shunt device 100′ surrounds at least a portion of delivery device 1100, and shunt device 100′ is configured to be coupled to delivery device 1100 for delivery into a human body. Further, the features and components of delivery device 1100 are configured to release shunt device 100′ from delivery device 1100 and allow shunt device 100′ to be coupled to tissue within the human body. To avoid any adverse effects of inserting shunt device 100′ and delivery device 1100 into a human body, both shunt device 100′ and delivery device 1100 can be sterilized before entering the human body.

In the example shown, central flow tube 110′ of shunt device 100′ surrounds a portion of molded bridge 1104, such that a portion of molded bridge 1104 extends through central flow tube 110′. Further, a portion of central flow tube 110′ is positioned adjacent and abutting a portion of pocket 1114 of molded bridge 1104. The portion of central flow tube 110′ abutting pocket 1114 and an arm of shunt device 100′ are coupled to the moveable-arm wire extending through fourth lumen 1116D, such that the moveable-arm wire couples shunt device 100′ to delivery device 1100. Further, the moveable-arm wire can be released from the portion of central flow tube 110′ abutting pocket 1114 and an arm of shunt device 100′ to allow shunt device 100′ to release from delivery device 1100 and couple to tissue within the human body.

Sensor 150′ is also positioned adjacent and abutting a portion of pocket 1114 of molded bridge 1104. More specifically, sensor 150′ extends from an arm of shunt device 100′ in a direction towards distal end 1101B of delivery device 1100 and sensor 150′ is positioned at least partially within pocket 1114 of molded bridge 1104. As shown, distal end 1104B of molded bridge 1104 and proximal end 1108A of tapered portion 1108 of nosecone 1102 include an enlarged diameter compared to a diameter or size of the other components and features of delivery device 1100. The enlarged diameter of distal end 1104B of molded bridge 1104 and proximal end 1108A of tapered portion 1108 is sized to be greater than the outermost point of sensor 150′ when sensor 150′ is positioned within pocket 1114 of molded bridge 1104 and surrounded by an outer sheath for insertion. As such, the enlarged diameter of distal end 1104B of molded bridge 1104 and proximal end 1108A of tapered portion 1108 is configured to enlarge a hole or aperture within tissue in the human body to allow sensor 150′ and shunt device 100′ to proceed through the hole or aperture within the tissue without inducing excess stress on the tissue. Therefore, the enlarged diameter of distal end 1104B of molded bridge 1104 and proximal end 1108A of tapered portion 1108 facilitates the easy insertion of shunt device 100′ and sensor 150′ into the human body. If distal end 1104B of molded bridge 1104 and proximal end 1108A of tapered portion 1108 did not include the enlarged portion, undesirable stresses could be induced in the tissue when inserting shunt device 100′ and sensor 150′ into the human body.

Terminal end 134A′ of distal arm 130A′ of shunt device 100′ is positioned adjacent and abutting a portion of notch 1112 of molded bridge 1104. Further, terminal end 134A′ is coupled to release wire 1124 (shown in FIGS. 11-12) extending through third lumen 1116C, such that release wire 1124 couples terminal end 134A′ and shunt device 100′ to delivery device 1100. More specifically, release wire 1124 extends through third lumen 1116C and an aperture within terminal end 134A′, securing terminal end 134A′ and shunt device 100′ to delivery device 1100. To release terminal end 134A′ and shunt device 100′ from delivery device 1100, release wire 1124 can be pulled through third lumen 1116C in the direction towards proximal end 1101A of delivery device 1100. Once release wire 1124 has been pulled through the aperture within terminal end 134A′, terminal end 134A′ and shunt device 100′ are released from delivery device 1100 which allows shunt device 100′to couple to tissue within the human body.

In operation, delivery device 1100 can be utilized to transport and place shunt device 100′ with the attached sensor 150′ within a human body, and then delivery device 1100 can be removed from the human body. Although the following discussion specifically describes shunt device 100′ with sensor 150′ coupled to delivery device 1100, it is to be understood that the following discussion equally applies to shunt device 100 (without a sensor) coupled to delivery device 1100. To transport and place shunt device 100′ in the human body, a guidewire is first inserted into the human body. Then the other end of the guidewire is inserted through the apertures extending through both straight portion 1106 and tapered portion 1108, and the guidewire is inserted into and extends through first lumen 1116A of molded bridge 1104. As such, delivery device 1100 is translated into and out of a human body by following the path and curvature of the guidewire. Delivery device 1100 is constructed from a soft, flexible material and therefore the entire delivery device 1100 curves as it is inserted and retracted from the human body. Further, in some examples, delivery device 1100 with an attached shunt device 100′ can be fully surrounded and covered by an outer sheath (not shown) to prevent delivery device 1100 with an attached shunt device 100′ and sensor 150′ from contacting, catching, or damaging tissue during the translation into the human body. After delivery device 1100 is inserted into the human body, the outer sheath (not shown) can be removed to uncover and expose shunt device 100′.

During the translation of delivery device 1100 into the human body, tapered portion 1108 of nosecone 1102 is configured to further dilate an aperture or opening within tissue in the human body to allow the downstream components of delivery device 1100 and shunt device 100′ to pass through the aperture or opening. As such, tapered portion 1108 of nosecone 1102 includes a larger diameter than the other components or features of delivery device 1100 to increase a diameter of an aperture within the tissue to facilitate the translation of delivery device 1100 into the human body. Once delivery device 1100 is translated into the human body, an outer sheath (not shown) covering and surrounding delivery device 1100 and shunt device 100′ can be pulled in a direction towards proximal end 1101A to uncover and expose shunt device 100′. Uncovering or exposing shunt device 100′ allows at least one arm of shunt device 100′ to release and extend outwards for seating on tissue within the human body. A contrast agent/die can then be injected through second lumen 1116B and into the human body, allowing a doctor or user to view the location of shunt device 100′ within the human body to ensure shunt device 100′ is properly positioned within the human body. In some examples, injecting the contrast agent/die comprises the contrast agent/die flowing from proximal end 1104A of molded bridge 1104, through second lumen 1116B, and then out through an aperture extending through body 1110 of molded bridge 1104 adjacent pocket 1114 of molded bridge 1104.

After verifying that the positioning of shunt device 100′ within the human body is correct, release wire 1124 can be removed from third lumen 1116C to allow shunt device 100′ to uncouple from delivery device 1100. More specifically, release wire 1124 can be pulled in a direction away from distal end 1101B of delivery device 1100 to pull release wire 1124 through third lumen 1116C until release wire 1124 is pulled past notch 1112 and no longer extends through an aperture within terminal end 134A′. This allows distal arm 130A′ and shunt device 100′ to release from delivery device 1100 and clamp onto tissue within the human body. Likewise, a moveable-arm wire (not shown) extending through fourth lumen 1116D can be removed from fourth lumen 1116D to allow shunt device 100′ to uncouple from delivery device 1100. More specifically, the moveable-arm wire can be pulled in a direction away from distal end 1101B of delivery device 1100 to pull the moveable-arm wire through fourth lumen 1116D until the moveable-arm wire no longer holds an arm and a portion of central flow tube 110′ of shunt device 100′ in contact with molded bridge 1104 of delivery device. This allows the arm and the portion of central flow tube 110′ of shunt device 100′ in contact with molded bridge 1104 to release from delivery device 1100 and clamp onto tissue within the human body.

After each of the arms of shunt device 100′ are released from delivery device 1100 and clamped onto tissue within the human body, delivery device 1100 can translate along the guidewire to remove delivery device 1100 from the human body. As such, a doctor or user can translate delivery device 1100 and shunt device 100′ with sensor 150′ into and out of the human body by pushing or pulling, respectively, on proximal end 1101A of delivery device 1100. Delivery device 1100 is an example device that is utilized to aid in transporting and placing shunt device 100′ within the human body. Placing or securing shunt device 100′ within the human body can include at least one arm of shunt device 100′ clamping onto tissue within the human body to fixedly couple shunt device 100′ within the human body. In some examples, shunt device 100′ can be clamped or coupled onto tissue within a heart of the human body to ensure an aperture or opening within the heart remains open after delivery device 1100 is removed from the human body.

FIG. 14 is a side view of distal portion 1101′ of delivery device 1100′. Delivery device 1100′ is substantially similar to delivery device 1100 with the exception that portions of first lumen 1116A′, configured to receive a guidewire, are eliminated from delivery device 1100′ to reduce a diameter of molded bridge 1104′. Distal portion 1101′ (having proximal end 1101A′ and distal end 1101B′), nosecone 1102′, molded bridge 1104′ (having proximal end 1104A′ and distal end 1104B′), straight portion 1106′, tapered portion 1108′ (having proximal end 1108A′ and distal end 1108B′), body 1110′, notch 1112′, pocket 1114′, vertical portion 1118′, horizontal portion 1120′, angled portion 1122′, first lumen 1116A′, and guidewire 1126 are shown. Delivery device 1100′ includes a plurality of lumens 1116 (not shown) substantially similar to second lumen 1116B, third lumen 1116C, and fourth lumen 1116D of delivery device 1100. The location of second lumen 1116B, third lumen 1116C, and fourth lumen 1116D of delivery device 1100′ can be modified with the elimination of lumen 1116A′. In contrast to delivery device 1100, first lumen 1116A′ extends through only a portion of body 1110′ of molded bridge 1104′. Nosecone 1102′, straight portion 1106′, and tapered portion 1108′ can be substantially the same as the corresponding features of delivery device 1100.

The complete or partial elimination of first lumen 1116A′ from body 1110′ of molded bridge 1104′ allows molded bridge 1104′ to be formed with a reduced total diameter as compared to molded bridge 1104 of delivery device 1100, which can be advantageous for some applications. Specifically, elimination of first lumen 1116A′ from a portion of body 1110A′ in which pocket 1114′ is formed can reduce a diameter of body 1110′ in the location of sensor 150′ thereby providing additional space for sensor 150′. As illustrated in FIG. 14, guidewire 1126 can be disposed along an exterior surface of body 1110′ of molded bridge 1104′. In some examples, guidewire 1126 can be disposed along an external surface of body 1110′ from proximal end 1104A′ to a distal end of pocket 1114′. Molded bridge 1104′ can include first lumen 1116A′ adjacent to distal end 1104B′ and extending between pocket 1114′ and distal end 1104B′ to capture guidewire 1126. First lumen 1116A′ extends through a portion of molded bridge 1104′ adjacent distal end 1104B′ of molded bridge 1104′ and is open to pocket 1114. First lumen 1116A′ is configured to accept and receive guidewire 1126 fully extending through first lumen 1116A′. Guidewire 1126 can extend through lumen 1116A′ and into and through nose cone 1102, exiting delivery device 1100′ at distal end 1101B′.

The distal end of first lumen 1116A′ at distal end 1104B′ of molded bridge 1104 is axially aligned with the apertures (not shown) extending through both straight portion 1106′ and tapered portion 1108′ of nosecone 1102′. In some examples, first lumen 1116A′ can include a curve or bend along the length of first lumen 1116A′ such that the proximal end of first lumen 1116A′ adjacent to pocket 1114 is not axially aligned with the distal end of first lumen 1116A′ at distal end 1104B′.

In alternative examples, first lumen 1116A′ can additionally extend through proximal end 1104A′ of molded bridge 1104 to pocket 1114′. As such, first lumen 1116A′ can be interrupted with a break or gap in first lumen 1116A′ along pocket 1114′. A proximal end of first lumen 1116A′ at proximal end 1104A′ of molded bridge 1104 can be axially aligned with a lumen extending through an inner sheath (not shown) of delivery device 1100′.

In some examples, all or portions of molded bridge 1104′ can be constructed from a metal, including but not limited to nitinol. In some examples, the portion of body 1110′ in which pocket 1114′ is formed can be formed of a metal and the portions of molded bridge 1104′ forming proximal end 1104A′ and distal end 1104B′ and through which one or more lumen (first lumen 1116A′, second lumen 1116B, third lumen 1116C, and fourth lumen 1116D) are disposed can be constructed from a thermoplastic elastomer, a nylon, or other polymer blend material.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Embodiments

The following are non-exclusive descriptions of possible embodiments of the present invention.

A delivery device for delivering a shunt device within a human body, the delivery device includes a nosecone including a straight portion and a tapered portion, and a molded bridge coupled to the nosecone. The molded bridge includes a body, a notch extending into the body of the molded bridge, and a pocket extending into the body of the molded bridge. The pocket is positioned on an opposite side of the molded bridge as the notch. The molded bridge further includes a plurality of lumens positioned within and extending at least partially through the body of the molded bridge.

The delivery device of the preceding paragraph can optionally include, additionally and/or alternatively, any one or more of the following features, configurations and/or additional components:

In an embodiment of the foregoing delivery device, the straight portion of the nosecone and the tapered portion of the nosecone can each include an aperture extending fully through the straight portion and the tapered portion, respectively. The aperture extending through the straight portion and the aperture extending through the tapered portion can be axially aligned and can be configured to accept a guidewire.

In an embodiment of any of the foregoing delivery devices, the tapered portion of the nosecone can include a distal end coupled to the straight portion of the nosecone, and wherein the tapered portion of the nosecone can include a proximal end coupled to a distal end of the molded bridge.

In an embodiment of any of the foregoing delivery devices, the proximal end of the tapered portion includes a first outer diameter and the distal end of the tapered portion can include a second outer diameter, and wherein the first outer diameter of the proximal end can be greater than the second outer diameter of the distal end.

In an embodiment of any of the foregoing delivery devices, the first outer diameter can range between 3 millimeters (0.118 inches) and 9 millimeters (0.354 inches).

In an embodiment of any of the foregoing delivery devices, the distal end of the molded bridge coupled to the proximal end of the tapered portion can include an outer diameter equal to the first outer diameter of the proximal end of the tapered portion.

In an embodiment of any of the foregoing delivery devices, the notch of the molded bridge can be positioned adjacent a proximal end of the molded bridge and configured to seat an arm of the shunt device.

In an embodiment of any of the foregoing delivery devices, the notch of the molded bridge can be positioned adjacent a proximal end of the molded bridge on a top side of the molded bridge, and wherein the notch can be configured to seat an arm of the shunt device.

In an embodiment of any of the foregoing delivery devices, the notch can include a vertical portion, a horizontal portion, and an angled portion.

In an embodiment of any of the foregoing delivery devices, the notch can include a vertical portion, a horizontal portion, and an angled portion, wherein the horizontal portion can extend between and connect the vertical portion to the angled portion.

In an embodiment of any of the foregoing delivery devices, the vertical portion can be positioned closest to a proximal end of the molded bridge compared to the horizontal portion and the angled portion, and wherein the vertical portion extends a partial distance from an outer surface of the molded bridge towards a central axis of the molded bridge.

In an embodiment of any of the foregoing delivery devices, the horizontal portion can extend between the vertical portion and the angled portion, and wherein the horizontal portion can extend from an edge of the vertical portion towards a distal end of the molded bridge, connecting the vertical portion to the angled portion.

In an embodiment of any of the foregoing delivery devices, the angled portion can be positioned closest to a distal end of the molded bridge compared to the horizontal portion and the vertical portion, and wherein the angled portion can extend from an end of the horizontal portion at an oblique angle towards the distal end of the molded bridge.

In an embodiment of any of the foregoing delivery devices, the pocket of the molded bridge can extend a partial distance between a proximal end of the molded bridge and a distal end of the molded bridge and can be configured to seat an arm and a portion of a central flow tube of the shunt device, and wherein the pocket can be further configured to seat a sensor coupled to the shunt device.

In an embodiment of any of the foregoing delivery devices, the pocket of the molded bridge can extend a partial distance between a proximal end of the molded bridge and a distal end of the molded bridge, and wherein the pocket can be positioned on a bottom side of the molded bridge opposite the notch positioned on a top side of the molded bridge.

In an embodiment of any of the foregoing delivery devices, the pocket can be configured to seat an arm and a portion of a central flow tube of the shunt device, and wherein the pocket can be configured to seat a sensor coupled to the shunt device.

In an embodiment of any of the foregoing delivery devices, the pocket can be a semi-circular indent extending towards a central axis of the molded bridge.

In an embodiment of any of the foregoing delivery devices, the pocket can be a semi-circular indent extending from a bottom side of the molded bridge towards a central axis of the molded bridge.

In an embodiment of any of the foregoing delivery devices, each of the plurality of lumens can be tubular channels extending at least partially through the body of the molded bridge.

In an embodiment of any of the foregoing delivery devices, each of the plurality of lumens can have differing lengths.

In an embodiment of any of the foregoing delivery devices, the plurality of lumens can be tubular channels of differing lengths.

In an embodiment of any of the foregoing delivery devices, the plurality of lumens can include a first lumen, a second lumen, a third lumen, and a fourth lumen.

In an embodiment of any of the foregoing delivery devices, the first lumen can fully extend from a proximal end of the molded bridge to a distal end of the molded bridge.

In an embodiment of any of the foregoing delivery devices, the first lumen can be configured to accept and receive a guidewire fully extending through the first lumen.

In an embodiment of any of the foregoing delivery devices, the plurality of lumens can include a first lumen, the first lumen can fully extend from a proximal end of the molded bridge to a distal end of the molded bridge and can be configured to accept and receive a guidewire fully extending through the first lumen.

In an embodiment of any of the foregoing delivery devices, the second lumen can extend from a proximal end of the molded bridge to the pocket.

In an embodiment of any of the foregoing delivery devices, the second lumen can be configured to provide a flow path for contrast dye or a contrast agent to flow through for delivery.

In an embodiment of any of the foregoing delivery devices, the plurality of lumens can include a second lumen, the second lumen can extend from a proximal end of the molded bridge to the pocket and can be configured to provide a flow path for contrast dye or a contrast agent to flow through for delivery.

In an embodiment of any of the foregoing delivery devices, the third lumen can extend from a proximal end of the molded bridge to the notch, and wherein the third lumen can include a break and then the third lumen resumes on an opposite side of the notch.

In an embodiment of any of the foregoing delivery devices, the third lumen can be configured to accept and receive a release wire extending within and at least partially through the third lumen.

In an embodiment of any of the foregoing delivery devices, the third lumen can extend through a vertical portion of the notch, and wherein the third lumen can extend through an angled portion of the notch.

In an embodiment of any of the foregoing delivery devices, the plurality of lumens can include a third lumen, the third lumen can extend from a proximal end of the molded bridge to the notch, and wherein the third lumen can include a break and then the third lumen can resume on an opposite side of the notch. The third lumen can be configured to accept and receive a release wire extending within and at least partially through the third lumen.

In an embodiment of any of the foregoing delivery devices, the fourth lumen can extend from a proximal end of the molded bridge to the pocket.

In an embodiment of any of the foregoing delivery devices, the fourth lumen can be configured to accept and receive a moveable-arm wire fully extending through the fourth lumen.

In an embodiment of any of the foregoing delivery devices, the plurality of lumen can include a fourth lumen, the fourth lumen can extend from a proximal end of the molded bridge to the pocket and can be configured to accept and receive a moveable-arm wire fully extending through the fourth lumen.

In an embodiment of any of the foregoing delivery devices, each of the first lumen, the second lumen, the third lumen, and the fourth lumen can be individually surrounded by an outer casing.

In an embodiment of any of the foregoing delivery devices, each of the outer casings can be constructed from a material having a higher durometer than the material used to construct the body of the molded bridge.

In an embodiment of any of the foregoing delivery devices each lumen of the plurality of lumens can be individually surrounded by an outer casing, and each of the outer casings can be constructed from a material having a higher durometer than the material used to construct the body of the molded bridge.

In an embodiment of any of the foregoing delivery devices, the nosecone can be constructed from one or more of a thermoplastic elastomer, a nylon, or other polymer blend material.

In an embodiment of any of the foregoing delivery devices, the body of the molded bridge can be constructed from one or more of a thermoplastic elastomer, a nylon, or other polymer blend material.

In an embodiment of any of the foregoing delivery devices, each of the nosecone and the body of the molded bridge can be constructed from one or more of a thermoplastic elastomer, a nylon, or other polymer blend material.

In an embodiment of any of the foregoing delivery devices, a distal end of a distal portion of the delivery device can be constructed from a polymer having a first durometer hardness and a proximal end of the distal portion of the delivery device can be constructed from a polymer having a second durometer hardness, and wherein the first durometer hardness can be less than the second durometer hardness.

In an embodiment of any of the foregoing delivery devices, the delivery device can be sterilized before entering the human body.

A method of using a delivery device to deliver a shunt device within a human body includes inserting a guidewire into the human body, and inserting an end of the guidewire positioned outside the human body into an aperture extending through a nosecone of the delivery device and a guidewire lumen of a molded bridge of the delivery device. The delivery device is translated along the guidewire to position the delivery device within the human body. A moveable-arm wire is removed from a moveable-arm lumen extending through the molded bridge to release a first proximal arm of the shunt device from the delivery device. A release wire is removed from a release wire lumen extending through the molded bridge to release a second proximal arm of the shunt device from the delivery device.

The method of the preceding paragraph can optionally include, additionally and/or alternatively, any one or more of the following features, configurations, additional components, and/or steps:

In an embodiment of the foregoing method, inserting an end of the guidewire positioned outside the human body into an aperture extending through a nosecone of the delivery device and a guidewire lumen of a molded bridge of the delivery device can further include inserting the guidewire into an aperture extending through a straight portion of a nosecone, inserting the guidewire into an aperture extending through a tapered portion of the nosecone, and inserting the guidewire into the guidewire lumen extending through a body of the molded bridge. The aperture extending through the straight portion of the nosecone and the aperture extending through the tapered portion of the nosecone can be axially aligned with the guidewire lumen extending through the body of the molded bridge.

An embodiment of any of the foregoing methods can further include dilating, by a tapered portion of the nosecone, an opening extending through a tissue wall to increase a diameter of the opening.

In an embodiment of any of the foregoing methods, the opening can dilate to a diameter ranging between 3 millimeters (0.118 inches) and 9 millimeters (0.354 inches).

An embodiment of any of the foregoing methods can further include translating the delivery device along the guidewire to remove the delivery device from the human body after the shunt device has been uncoupled from the delivery device.

An embodiment of any of the foregoing methods can further include injecting a contrast dye into the human body through a contrast lumen extending through a body of the molded bridge to verify a correct positioning of the shunt device within the human body.

In an embodiment of any of the foregoing methods, injecting the contrast dye can further include flowing the contrast dye from a proximal end of the contrast lumen, through the contrast lumen, and then out through an aperture extending through the body of the molded bridge adjacent a pocket of the molded bridge.

In an embodiment of any of the foregoing methods, removing the release wire from the release wire lumen can include pulling the release wire proximally away from a distal end of the delivery device.

In an embodiment of any of the foregoing methods, removing a release wire from a release wire lumen extending through the molded bridge to release a second proximal arm of the shunt device from the delivery device can further include pulling the release wire proximally until a distal end of the release wire translates past a notch in the body of the molded bridge to release the second proximal arm of the shunt device.

In an embodiment of any of the foregoing methods, removing a moveable-arm wire from a moveable-arm lumen extending through the molded bridge to release a first proximal arm of the shunt device from the delivery device can further include pulling the moveable-arm wire proximally until a distal end of the moveable-arm wire is released from the first proximal arm of the shunt device.

In an embodiment of any of the foregoing methods, pulling the moveable-arm wire proximally until a distal end of the moveable-arm wire is released from the first proximal arm of the shunt device can allow the first proximal arm of the shunt device to secure the shunt device within the human body.

In an embodiment of any of the foregoing methods, securing the shunt device within the human body can include clamping the first proximal arm of the shunt device onto a tissue wall.

In an embodiment of any of the foregoing methods, the tissue wall the shunt device is clamped onto can be between a coronary sinus and a left atrium of a heart.

In an embodiment of any of the foregoing methods, the guidewire lumen can fully extend from a proximal end of the molded bridge to a distal end of the molded bridge.

In an embodiment of any of the foregoing methods, a contrast lumen can extend from a proximal end of the molded bridge to a pocket.

In an embodiment of any of the foregoing methods, the release wire lumen can extend from a proximal end of the molded bridge to a notch, and wherein the release wire lumen can include a break and then the release wire lumen resumes on an opposite side of the notch.

In an embodiment of any of the foregoing methods, wherein the moveable-arm lumen can extend from a proximal end of the molded bridge to a pocket.

An embodiment of any of the foregoing methods can further include sterilizing the delivery device before translating the delivery device into the human body.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A delivery device for delivering a shunt device within a human body, the delivery device comprising:
a nosecone including a straight portion and a tapered portion;
a molded bridge coupled to the nosecone, the molded bridge comprising:
a body;
a notch extending into a first side of the body of the molded bridge; and
a plurality of lumens positioned within and extending at least partially through the body of the molded bridge, wherein the plurality of lumens are tubular channels of differing lengths.

2. The delivery device of claim 1, wherein the notch of the molded bridge is positioned adjacent a proximal end of the molded bridge, and wherein the notch is configured to seat an arm of the shunt device.

3. The delivery device of claim 1, wherein the notch includes a vertical portion, a horizontal portion, and an angled portion, wherein the horizontal portion extends between and connects the vertical portion to the angled portion.

4. The delivery device of claim 3, wherein the vertical portion is positioned closest to a proximal end of the molded bridge compared to the horizontal portion and the angled portion, and wherein the vertical portion extends a partial distance from an outer surface of the molded bridge towards a central axis of the molded bridge.

5. The delivery device of claim 4, wherein the angled portion is positioned closest to a distal end of the molded bridge compared to the horizontal portion and the vertical portion, and wherein the angled portion extends from an end of the horizontal portion at an oblique angle towards the distal end of the molded bridge.

6. The delivery device of claim 1, and further comprising a pocket extending into a second side of the body of the molded bridge opposite the notch, wherein the pocket of the molded bridge extends a partial distance between a proximal end of the molded bridge and a distal end of the molded bridge and is configured to seat an arm and a portion of a central flow tube of the shunt device, and wherein the pocket is configured to seat a sensor coupled to the shunt device.

7. The delivery device of claim 6, wherein the pocket is a semi-circular indent extending towards a central axis of the molded bridge.

8. The delivery device of claim 1, wherein the plurality of lumens includes a guidewire lumen, the guidewire lumen fully extending from a proximal end of the molded bridge to a distal end of the molded bridge and configured to accept and receive a guidewire fully extending through the guidewire lumen.

9. The delivery device of claim 1, wherein the plurality of lumens includes a contrast lumen, the contrast lumen extending from a proximal end of the molded bridge to a pocket and configured to provide a flow path for contrast dye or a contrast agent to flow through for delivery.

10. The delivery device of claim 1, wherein the plurality of lumens includes a release wire lumen, the release wire lumen extending from a proximal end of the molded bridge to the notch, and wherein the release wire lumen includes a break and then the release wire lumen resumes on an opposite side of the notch, the release wire lumen configured to accept and receive a release wire extending within and at least partially through the release wire lumen on opposite sides of the notch.

11. The delivery device of claim 1, wherein the plurality of lumens includes a moveable arm lumen, the moveable arm lumen extending from a proximal end of the molded bridge to a pocket and configured to accept and receive a moveable-arm wire fully extending through the moveable arm lumen.

12. The delivery device of claim 1, wherein each lumen of the plurality of lumens is individually surrounded by an outer casing, and wherein each of the outer casings is constructed from a material having a higher durometer than the material used to construct the body of the molded bridge.

13. A delivery device for delivering a shunt device within a human body, the delivery device comprising:
a nosecone including a straight portion and a tapered portion, wherein the tapered portion of the nosecone includes a proximal end coupled to a distal end of a molded bridge, the proximal end of the tapered portion including a first outer diameter, and wherein the tapered portion of the nosecone includes a distal end coupled to the straight portion of the nosecone, the distal end of the tapered portion including a second outer diameter, wherein the first outer diameter of the proximal end is greater than the second outer diameter of the distal end;
wherein the molded bridge includes a body, and a plurality of lumens positioned within and extending at least partially through the body of the molded bridge.

14. The delivery device of claim 13, wherein:
the straight portion of the nosecone and the tapered portion of the nosecone each include an aperture extending fully through the straight portion and the tapered portion, respectively;
the aperture extending through the straight portion and the aperture extending through the tapered portion are axially aligned; and
the aperture extending through the straight portion and the aperture extending through the tapered portion are configured to accept a guidewire.

15. The delivery device of claim 13, wherein each of the nosecone and the body of the molded bridge is constructed from one or more of a thermoplastic elastomer, a nylon, or other polymer blend material.

16. The delivery device of claim 13, wherein a distal end of a distal portion of the delivery device is constructed from a polymer having a first durometer hardness and a proximal end of the distal portion of the delivery device is constructed from a polymer having a second durometer hardness, and wherein the first durometer hardness is less than the second durometer hardness.

17. A delivery device for delivering a shunt device within a human body, the delivery device comprising:
a nosecone including a straight portion and a tapered portion;
a molded bridge coupled to the nosecone, the molded bridge comprising:
a body;
a notch extending into a first side of the body of the molded bridge; and
a plurality of lumens positioned within and extending at least partially through the body of the molded bridge, the plurality of lumens including a release wire lumen, the release wire lumen extending from a proximal end of the molded bridge to the notch, and wherein the release wire lumen includes a break and then the release wire lumen resumes on an opposite side of the notch, the release wire lumen configured to accept and receive a release wire extending within and at least partially through the release wire lumen on opposite sides of the notch.

18. The delivery device of claim 17, wherein the plurality of lumens are tubular channels of differing lengths.

19. The delivery device of claim 17, wherein the plurality of lumens includes:
a guidewire lumen, the guidewire lumen fully extending from a proximal end of the molded bridge to a distal end of the molded bridge and configured to accept and receive a guidewire fully extending through the guidewire lumen.

20. The delivery device of claim 17, wherein the plurality of lumens includes:
a contrast lumen, the contrast lumen extending from a proximal end of the molded bridge to a pocket and configured to provide a flow path for contrast dye or a contrast agent to flow through for delivery; and
a release wire lumen, the release wire lumen extending from a proximal end of the molded bridge to the notch, and wherein the release wire lumen includes a break and then the release wire lumen resumes on an opposite side of the notch, the release wire lumen configured to accept and receive a release wire extending within and at least partially through the release wire lumen.
